(19) United States
(12) Patent Application Publication
(10) Pub. No.: US 2010/0179512 A1
(43) Pub. Date: Jul. 15, 2010

(54) CATHETER STEERING/INSERTION MECHANISM

(76) Inventors: Evan Chong, South Strathfied (AU); Jesse Woolaston, Cremorne (AU)

Correspondence Address: MORRISON & FOERSTER LLP, 755 PAGE MILL RD, PALO ALTO, CA, 94304-1018, US

(21) Appl. No.: 12/516,760

(22) PCT Filed: Oct. 29, 2007
(86) PCT No.: PCT/AU07/01638

§ 371 Date: Mar. 2, 2010

Related U.S. Application Data
(60) Provisional application No. 60/861,773, filed on Nov. 28, 2006.

(51) Int. Cl. A61M 25/09

(52) U.S. Cl. 604/528

(57) ABSTRACT

A catheter steering/insertion mechanism (10) includes an elongate element (12) insertable into a lumen of a catheter sheath to assist in imparting a predetermined shape (14) to a distal region of the catheter sheath A control element (16) is mounted co-axially about the elongate element (12). The control element (16) is flexible in bending in a first configuration and resistant to bending in a second configuration so that the predetermined shape (14) is imparted to a distal region of the elongate element (12) and, in turn, the distal region of the catheter sheath when the control element (14) is in its first configuration.

### CROSS-REFERENCE TO RELATED APPLICATIONS

The present application claims priority from U.S. of America Provisional Patent Application No. 60/861,773 filed on 28 Nov. 2006, the contents of which are incorporated herein by reference.

### FIELD

This invention relates, generally, to a catheter and, more particularly, to a catheter steering/insertion mechanism and to a catheter including such catheter steering/insertion mechanism.

### BACKGROUND

A catheter to be used in treating cardiac disorders is conventionally inserted into a patient's vascular system via the femoral vein and a distal end of the catheter is steered through the vascular system to the site at the heart to be treated. To enable steering to be effected, the catheter has a steering mechanism.

A catheter manufactured in accordance with the Applicant's manufacturing technique as described in PCT/AU01/01339 dated 19 Oct. 2001 and entitled “An electrical lead” has the advantage that the electrode sheath of the catheter has an unimpeded lumen into which a steering mechanism can be inserted.

In certain applications, fixed curve stylets are used in place of a steering mechanism to access particular sites in the patient's body to be treated. There is a difficulty involved, firstly, in inserting such a stylet into the lumen of the electrode sheath and, secondly, in steering a catheter which has a curved end resulting from the stylet through the patient's vascular system.

### SUMMARY

According to the invention, there is provided a catheter steering/insertion mechanism which includes

an elongate element insertable into a lumen of a catheter sheath to assist in imparting a predetermined shape to a distal region of the catheter sheath, in use; and

a control element mounted co-axially about the elongate element, the control element being flexible in bending in a first configuration and resistant to bending in a second configuration so that the predetermined shape is imparted to a distal region of the elongate element and, in turn, to the distal region of the catheter sheath when the control element is in its first configuration.

At least a part of the control element in register, in use, with the distal end of the elongate element to which the predetermined shape is to be imparted may be helically coiled. At least a portion of coils of the part of control element may be spaced from each other when the control element is in its first configuration and the coils may be in abutment with one another when the control element is in its second configuration.

Preferably, the control element is a coil spring structure. If desired, control members may be carried by the coil spring structure for effecting manipulation of the coil spring structure between its first configuration and its second configuration.

The coil spring structure may be covered by a protective sheath. The protective sheath may be a sleeve of a heat shrink material.

The coils of the coil spring structure may be of rectangular (including square) cross-section to provide improved stability. Further, the coils, when viewed from a side of the coil spring structure, may have interlocking structures with the structures of adjacent coils interlocking when the control element is in its second configuration to enhance torsional stiffness of the control element. The interlocking structures may be sawtooth formations, sinusoidal formations, crenelated formations, or the like. This also has the added advantage that the overall length of the control element and, hence, the length of displacement of a control device on a catheter handle can be reduced. It will be appreciated that, by reducing the length of displacement of the control device, the length of the handle itself may be able to be reduced.

Still further, a cross-section of a length of material from which the coil spring is formed may vary periodically along its length. The arrangement may be such that, when coiled, parts of the same cross-sectional area are aligned so that, when the control element is in its second configuration a further, different shape is imparted to the catheter sheath.

In an embodiment, the elongate element may be of a superlastic alloy which has its distal region pre-formed into the predetermined shape, the control element being arranged about at least the distal region of the elongate element. In this embodiment, when the control element is in its first configuration, the distal region of the elongate element has the predetermined shape. When the control element is in its second configuration, the predetermined shape is eliminated.

The elongate element may be a steering assembly of which at least one component is of a superlastic alloy having its distal region pre-formed into the predetermined shape, the control element being arranged about at least the distal region of the steering assembly. The steering assembly may be as described in the Applicant's International Patent Application No. PCT/AU2005/000216 dated 18 Feb. 2005 and entitled “A steerable catheter”.

In another embodiment, the coils of the coil spring structure of the control element may abut along a predetermined line to have the predetermined shape. The coils may be connected to one another where they abut. The line may be a rectilinear, i.e. a straight, line. Instead, the line may spiral about the coil spring structure to impart the predetermined shape to the control element. It will be appreciated that, in this embodiment, the elongate element may merely act as a flexible pull wire and need not be of a superlastic alloy. However, the elongate element may be of the superlastic alloy with the predetermined shape to enhance shape formation.

In yet a further embodiment, the coil spring structure may be of a superlastic alloy preformed into the predetermined shape. Once again, in this embodiment, the elongate element may merely act as a flexible pull wire and need not be of a superlastic alloy. However, the elongate element may be of the superlastic alloy with the predetermined shape to enhance shape formation.

The invention extends also to a catheter which includes

an electrode sheath defining a lumen; and

a catheter steering/insertion mechanism, as described above, received within the lumen of the electrode sheath.

### BRIEF DESCRIPTION OF DRAWINGS

FIG. 1 shows a schematic, exploded view of a catheter steering/insertion mechanism, in accordance with an embodiment of the invention;

FIG. 2 shows a schematic, side view of the catheter steering/insertion mechanism;

FIG. 3 shows, on an enlarged scale, a schematic, side view of the circled part labelled ‘A’ in FIG. 2;

FIG. 4 shows a schematic, three dimensional view of an elongate element of the catheter steering/insertion mechanism;

FIG. 5 shows a schematic, side view of an embodiment of a control element of the catheter steering/insertion mechanism;

FIG. 6 shows a schematic, side view of another embodiment of a control element of the catheter steering/insertion mechanism; and

FIG. 7 shows, on an enlarged scale, a schematic, side view of the circled part labelled ‘B’ of the component of FIG. 6.

### DETAILED DESCRIPTION OF EXEMPLARY EMBODIMENTS

In the drawings, reference numeral 10 generally designates a catheter steering/insertion mechanism in accordance with an embodiment of the invention. The mechanism 10 comprises an elongate element 12 insertable into a lumen of a catheter sheath (not shown) to assist in imparting a predetermined shape 14 to a distal part of the catheter sheath, in use.

A control element 16 is coaxially mounted about the elongate element 12. The control element 16 is of a flexible construction and is flexible in bending in a first 10 configuration and is resistant to bending in a second configuration so that the predetermined shape 14 is imparted to the distal end of the elongate element 12 when the control element 16 is in its first configuration.

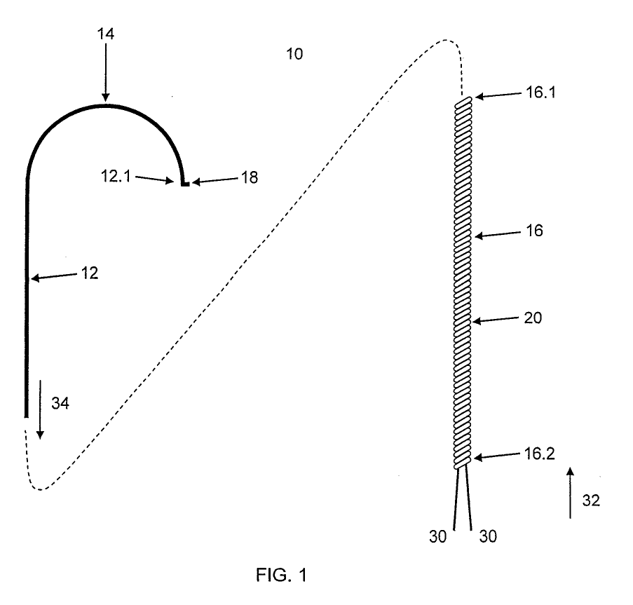

In the embodiment shown in FIG. 1 of the drawings, the elongate element 12 is a wire of a superlastic alloy. The wire 12 is heat set to impart the shape 14 to the distal end of the wire 12.

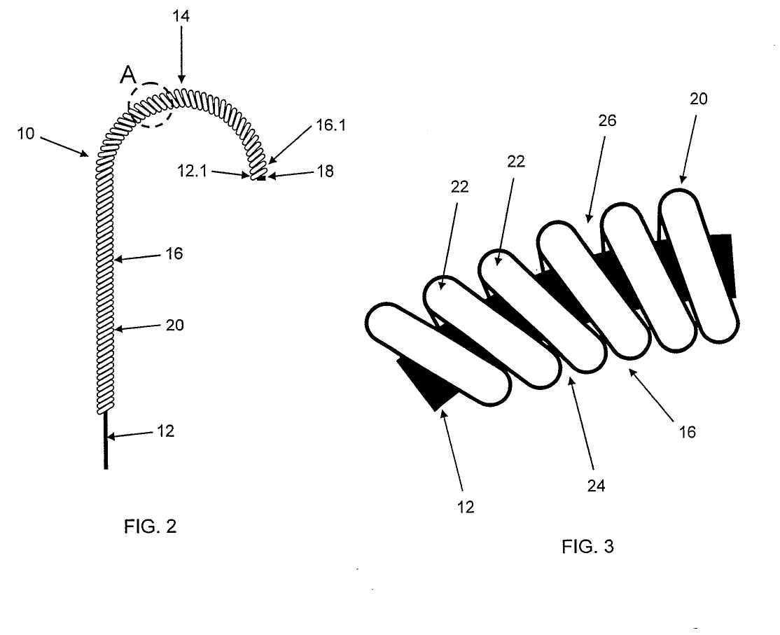

An anchor formation 18 is arranged at a distal end 12.1 of the wire 12 to which a distal end 16.1 of the control element 16 is anchored, in use, as shown in FIG. 2 of the drawings.

In the embodiment illustrated in FIGS. 1 to 3 of the drawings, the control element is in the form of a sleeve of a coil spring structure 20. The coil spring structure 20 has a plurality of spaced coils 22, at least a part of adjacent coils 22 being spaced from each other when the control element 16 is in its first configuration. This is shown in greater detail in FIG. 3 of the drawings. It will be appreciated that, where the spring follows the predetermined shape 14 of the wire 12, the coils 22 will bunch up on one side of the shape 14 as shown at 24 in FIG. 3 of the drawings. On the opposed side, the coils will move further apart as shown at 26.

The coil spring structure 20 is of a suitable biocompatible steel such as surgical grade stainless steel. The control element 16 can be of an off the shelf construction and no special machining or working of the control element 16 is required for this application.

It will be appreciated that, in its first configuration, the coils 22 are at least partially spaced apart from each other and, when the control element 16 is mounted about the wire 12, the shape 14 is extant. Conversely, when the coils 22 of the coil spring structure 20 of the control element 16 are urged together, the predetermined shape 14 is eliminated and the distal part of the wire 12 straightens out. This, firstly, facilitates insertion of the steering/insertion mechanism 10 into a lumen of an electrode sheath of a catheter manufactured in accordance with the Applicant's above-referenced international application. Also, while the coils 22 of the coil spring structure 20 are in abutment, the distal end of the electrode sheath is straight and this facilitates steering of the catheter through the vasculature of a patient's body.

To effect manipulation of the control element 16 between its first configuration and its second configuration, relative movement between the control element 16 and the wire 12 is required. This can be achieved in a number of ways. For example, the proximal end of the control element could carry control members as indicated by dotted lines 30 in FIG. 1 of the drawings. By pushing on the control members 30 in the direction of arrow 32, the coils 22 of the coil spring structure 20 can be brought into abutment with each other to straighten the predetermined shape 14. Conversely, by releasing the control members 30, the predetermined shape 14 is imparted to the distal part of the wire 12.

In another embodiment, a proximal end 16.2 of the control element 16 could be anchored within the lumen of the catheter sheath. By pulling on the wire 12 in the direction of arrow 34 relative to the control element 16, the coils 22 of the coil spring structure 20 can be brought into abutment with each other to straighten the predetermined shape 14. Conversely, by releasing the wire 12 the control element 16 is relaxed, the coils 22 of the coil spring structure 20 move apart and the shape 14 is imparted to the distal part of the wire 12.

To provide improved stability, a wire coiled to form the coil spring structure 20 is of square (including rectangular) cross-section. This also provides improved stability for the mechanism 10.

It will be readily understood that, as the coil spring structure 20 is manipulated, the predetermined shape 14 will change. Thus, as the coils 22 of the coil spring structure 20 move towards each other, a more gradual radius of curvature will be imparted to the predetermined shape 14. Thus, the actual shape of the predetermined shape 14 can be controlled by manipulation of the control element 16 relative to the wire 12.

In yet a further embodiment, the control element 16, itself, is fabricated of a superlastic alloy. In this embodiment, the predetermined shape is imparted to the control element 16 by heat setting it. The wire 12 then merely acts as a stiffener for the control element 16. The elimination or application of the predetermined shape in this embodiment is achieved in the same way by relative longitudinal displacement occurring between the control element 16 and the wire 12.

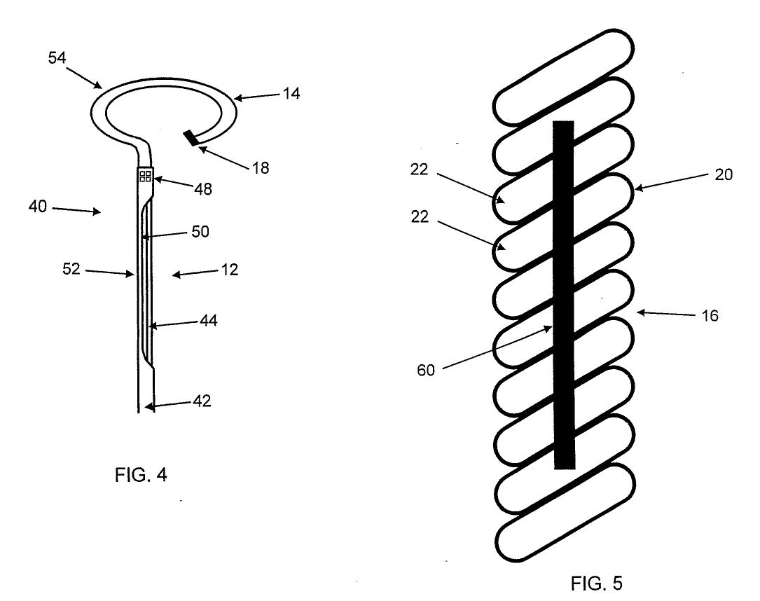

Referring to FIG. 4 of the drawings, another variation of the elongate element 12 of the insertion mechanism 10 is shown.

In this embodiment, the elongate element 12 constitutes a steering assembly 40 of the mechanism 10. The steering assembly 40 is of the type described in the Applicant's International Application No. PCT/AU2005/000216 dated 18 Feb. 2005 entitled “A steerable catheter”. Thus, the steering assembly 40 includes a tubular member 42 in which an actuator 44 is received. A part of the actuator 44 is fast with a distal end 46 of the tubular member 42, for example, by being crimped together, as shown at 48 in FIG. 4 of the drawings. The tubular member 42 has a cutaway portion 50 at a distal region, but proximally of the crimped zone 48, which forms a bend enhancing zone 52. Longitudinal movement of the tubular member 42 and the actuator 44 relative to each other causes bending of the tubular member 42 and, hence, the electrode sheath of the catheter in which the steering assembly 40 is inserted. The actuator 44 has a distal region 54 arranged distally of the crimped zone 48 which is bent into the predetermined shape, for example, a loop shape.

In this embodiment, at least the actuator 44 is of a heat set superlastic alloy which has its distal part 54 formed into the predetermined shape. The tubular member 42 can also be of the superlastic alloy.

In this embodiment, the control element 16 is placed over the distal part of the steering assembly 40 and, as described above with reference to FIGS. 1 to 3 of the drawings, by closing the coils 22 of the coil spring structure 20 relative to each other, the shape 14 at the distal part 54 of the actuator 44 is straightened out for insertion or steering of a catheter in which the mechanism 10 is inserted, in use.

This embodiment has the added advantage that the control element 16, in use, extends over the cutaway portion 50 of the tubular member 42. The control element 16 therefore acts as a cage about the portion of the actuator 44 in the cutaway portion 50 and serves to restrain the actuator 44 within the cutaway portion 50 of the tubular member 42 during bending about the cutaway portion 50.

In FIG. 5 of the drawings, another embodiment of the control element 16 is shown. In this embodiment, the coils 22 are secured together along a line 60. It will be appreciated that, by securing one side of the coils 22 together, the coil spring structure 20 has a predetermined, curved shaped imparted to it. A simple pull wire (not shown) can be mounted on a diametrically opposed location on the coils 22 to pull the coils 22 together to straighten out the predetermined shape. With this embodiment, the elongate element 12 may merely act as a stiffener or, instead, could also have the predetermined shape 14 imparted to it to enhance shape formation of the distal part of the catheter sheath, in use.

As a variation of this embodiment, the line 60 may not be a straight line as illustrated in FIG. 5 of the drawings. Instead, the line 60 may be arranged spirally on the coils 22 of the coil spring structure 20. This will impart a spiral-like shape to the distal part of the control element 16 which, in turn, is imparted, in use to the electrode sheath of the catheter.

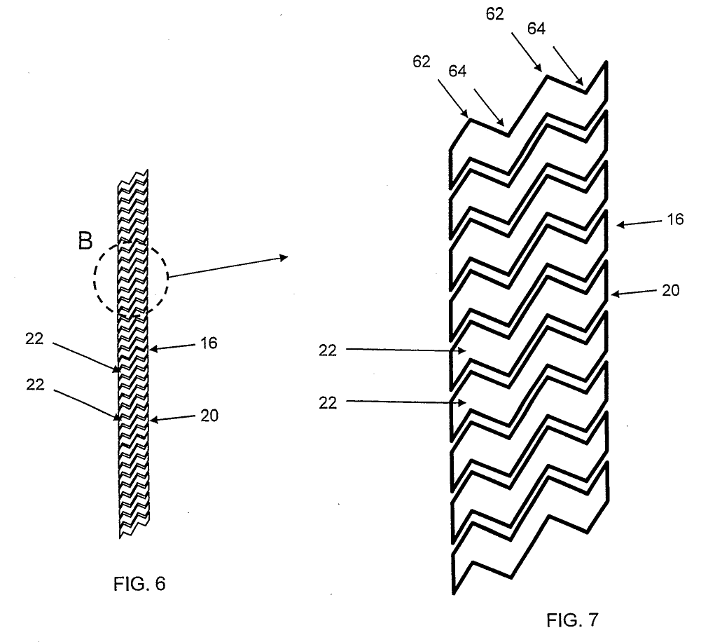

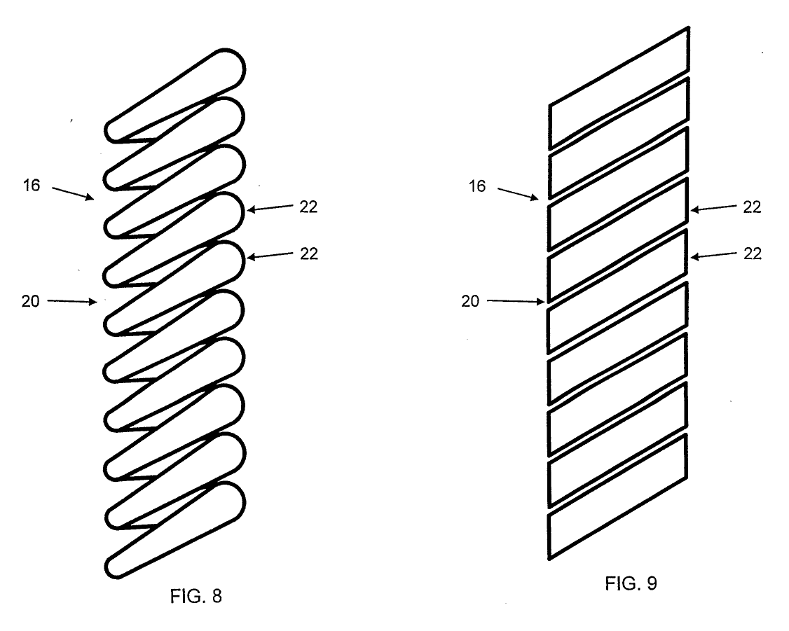

In FIGS. 6 and 7 of the drawings, yet a further embodiment of the control element 16 is illustrated. With reference to the previous drawings, like reference numerals refer to like parts, unless otherwise specified.

In this embodiment, the coils 22 of the coil spring 20 structure have a sawtooth shape, when viewed end on, as shown in greater detail in FIG. 7 of the drawings. This sawtooth shape defines a plurality of intercalating teeth 62 and valleys 64.

With this arrangement, when the coils 22 of the coil spring structure 20 are spaced apart from each other, the control element 16 is flexible, both in bending and in torsion. This allows the shape 14 to be imparted to the elongate element 12, as described above. However, when the coils 22 are brought into abutment with each other, the teeth 62 and valleys 64 of the coils 22 mesh providing both torsional rigidity and resistance to bending. This serves to straighten out the predetermined shape 14 and provides torsional stiffness to the mechanism 10.

An advantage of this embodiment of the invention is that a shorter length of the control element 16 can be used than would otherwise be the case. This allows a significantly shorter catheter handle to be use in that a displacement mechanism (not shown) for effecting manipulation of the control element 16 between its first configuration and its second configuration requires a shorter distance of travel.

It will be appreciated that, in this embodiment, the cross section of the coils 22 is, once again, square or rectangular. Also, instead of the sawtooth shape of the coils 22, the coils 22 could have other interlocking shapes, for example, crenelations, square or sinusoidal waveforms, or the like.

Hence, it is an advantage of the invention that a catheter steering/insertion mechanism is provided which is cheap to produce as there is no specialised tubing or machining required. The steering mechanism is flexible when the control element 16 is in its first configuration since the non-straightened stiffness of the mechanism 10 is, essentially, that of the wire 12.

An extremely simple but effective straightening mechanism is provided using the control element 16 of the mechanism 10.

Also, in the embodiment shown in FIG. 4 of the drawings, the control element 16 serves the additional purpose of acting as a cage which serves to restrain the actuator 44 within the tubular member 42 of the steering assembly 40 in the cutaway portion 50 of the tubular member 42.

Still further, a control element 16 which provides torsional stiffness at least in its second configuration is also provided which facilitates steering of the mechanism 10 and, accordingly, the catheter within which the steering mechanism 10 is inserted through the vascular system of a patient's body.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A catheter steering/insertion mechanism which includes
an elongate element insertable into a lumen of a catheter sheath to assist in imparting a predetermined shape to a distal region of the catheter sheath, in use; and
a control element mounted co-axially about the elongate element, the control element being flexible in bending in a first configuration and resistant to bending in a second configuration so that the predetermined shape is imparted to a distal region of the elongate element and, in turn, to the distal region of the catheter sheath when the control element is in its first configuration.

2. The mechanism of claim 1 in which at least a part of the control element in register, in use, with the distal region of the elongate element to which the predetermined shape is to be imparted is helically coiled.

3. The mechanism of claim 2 in which at least a portion of coils of the at least part of the control element are spaced from each other when the control element is in its first configuration and the coils are in abutment with one another when the control element is in its second configuration.

4. The mechanism of claim 3 in which the control element is a coil spring structure.

5. The mechanism of claim 4 in which control members are carried by the coil spring structure for effecting manipulation of the coil spring structure between its first configuration and its second configuration.

6. The mechanism of claim 4 in which the coils of the coil spring structure are of rectangular cross-section to provide improved stability.

7. The mechanism of claim 4 in which the coils, when viewed from a side of the coil spring structure, have interlocking structures with the structures of adjacent coils interlocking when the control element is in its second configuration to enhance torsional stiffness of the control element.

8. The mechanism of claim 1 in which the elongate element is of a superlastic alloy which has its distal region pre-formed into the predetermined shape, the control element being arranged about at least the distal region of the elongate element.

9. The mechanism of claim 8 in which the elongate element is a steering assembly of which at least one component is of a superlastic alloy having its distal region pre-formed into the predetermined shape, the control element being arranged about at least the distal region of the steering assembly.

10. The mechanism of claim 3 in which the coils of the coil spring structure of the control element abut along a predetermined line to have the predetermined shape.

11. The mechanism of claim 10 in which the line spirals about the coil spring structure to impart the predetermined shape to the control element.

12. The mechanism of claim 3 in which the coil spring structure is of a superlastic alloy preformed into the predetermined shape.
